(19) United States
(12) Patent Application Publication
(10) Pub. No.: US 2018/0321223 A1
(43) Pub. Date: Nov. 8, 2018

(54) Method for Monitoring the Immunological Profile of a Subject

(71) Applicants: INSERM (Institut National de la Sante et de la Recherche Medicale), Paris (FR); Centre National de la Recherche Scientifique - CNR, Paris (FR); Universite Paul Sabatier Toulouse III, Toulouse (FR); Universite Paris Diderot - Paris 7, Paris (FR)

(72) Inventors: Nabila Jabrane-Ferrat, Toulouse Cedex 3 (FR); Johan Siewiera, Toulouse Cedex 3 (FR); Reem Al-Daccak, Paris Cedex 10 (FR)

(21) Appl. No.: 15/772,319

(22) PCT Filed: Oct. 26, 2016
(86) PCT No.: PCT/EP2016/075783

§ 371 Date: Apr. 30, 2018

(30) Foreign Application Priority Data
Oct. 30, 2015 (EP) 15306735.0

(51) Int. Cl. G01N 33/50; C12Q 1/6886; A61P 31/18; A61P 31/16; C07K 16/24

(52) U.S. Cl. CPC G01N 33/5023; C12Q 1/6886; G01N 33/56966; A61P 31/16; C07K 16/244; A61P 31/18

(57) ABSTRACT

The invention relates to a method for monitoring the immunological profile of a subject, comprising measuring the expressions of each member of the group consisting of NKp30a, NKp30b, NKp30c, NKp44b and NKp44c, in a biological sample of said subject, wherein: if the expressions of NKp30a, NKp30b, and NKp44b are the three highest among said group, then said subject has a responsive profile; or if the expressions of NKp30c and NKp44c are the two highest among said group, then said subject has an unresponsive profile.

### FIELD OF THE INVENTION

The invention relates to a method for monitoring the immunological profile of a subject in need thereof, comprising measuring the expressions of each member of the group consisting of NKp30a, NKp30b, NKp30c, NKp44b and NKp44c, in a biological sample of said subject,

wherein:
if the expressions of NKp30a, NKp30b, and NKp44b are the three highest among said group, then said subject has a responsive profile; or
if the expressions of NKp30c and NKp44c are the two highest among said group, then said subject has an unresponsive profile.

### BACKGROUND OF THE INVENTION

Natural Killer (NK) cells are a pool of distinct innate immune cells that play a key role in controlling pathological situations such as viral infection or tumor and also more physiological ones such as pregnancy. NK cell effector functions are orchestrated by a wide array of germline-encoded receptors (NKR), expressed in stochastic pattern. Natural cytotoxicity receptors (NCR) are among the major NK cell activating receptors that recognize yet to be identified self-ligands. NCRs belong to the immunoglobulin-like family and are involved in NK cell cytotoxic function against infected cells and tumors.

Peripheral NK cells (pNK) and decidua basalis NK cells (dNK) from the pregnant uterus lining are two distinct subsets of the physiological NK cells pool. They are clearly different at both phenotypical and functional levels¹²⁻¹⁷. Very little is known about the origin of dNK cells as they could derive either from NK cell progenitors or mature pNK cells that migrate/proliferate/differentiate in a local environment enriched in steroids and cytokines/chemokines¹⁸⁻²⁰. At the functional level, dNK cells participate actively in fetal trophoblast differentiation/invasion and vascular remodeling that are mandatory for successful human pregnancy^{14,21,22}, whereas pNK are involved in the immune response against various threats.

It would be useful to monitor the immune system of a given subject, such as a subject suffering from an infection or a tumor. Indeed, such a monitoring would allow choosing the best therapeutic strategy for each subject, in view of the activity of his immune system.

There is thus a need for a monitoring of the immunological status of a given subject over time, which may be easy and quick to perform. Such a monitoring would have to be sensitive and specific, so as to determine whether the subject has an active immune system.

### SUMMARY OF THE INVENTION

The inventors have surprisingly discovered that the expression of alternatively spliced variants of NCR delineate the two NK cell subsets (pNK and dNK) and their differential functioning. By analyzing a cohort of dNK and pNK cells from the same donors, as shown in the example, they demonstrate that first trimester dNK cells express NCR isoforms that are different from those expressed by pNK cells, and this differential expression is physiologically relevant. Indeed, whereas dNK cells rather express NKp30c and NKp44c, pNK cells rather express NKp30a, NKp30b, and NKp44b. This difference in NCR isoforms expressions considerably impacts dNK lytic activity and is sculptured by the decidual cytokine microenvironment that selects for inhibitory rather than activating isoforms of NKp30 and NKp44.

Thus, the invention provides a method for monitoring the immunological profile of a subject in need thereof, comprising measuring the expressions of each member of the group consisting of NKp30a, NKp30b, NKp30c, NKp44b and NKp44c, in a biological sample of said subject,

wherein:
if the expressions of NKp30a, NKp30b, and NKp44b are the three highest among said group, then said subject has a responsive profile; or
if the expressions of NKp30c and NKp44c are the two highest among said group, then said subject has an unresponsive profile.

The invention also relates to a method for converting a sample of NK cells having a responsive profile into NK cells having an unresponsive profile, comprising a step of mixing said sample with a composition comprising TGF-β and IL-15.

Another aspect of the invention relates to a method for treating a subject grafted with cells or organ, comprising the following steps:

i) measuring the expressions of NKp30a, NKp30b, NKp30c, NKp44b and NKp44c in a biological sample of said subject;
if the expressions of NKp30a, NKp30b, and NKp44b are the three highest among said group, then said subject has a responsive profile; and
ii) treating said patient with an immunosuppressive drug.

Yet another aspect of the invention relates to a method for treating a subject grafted with cells or organ, comprising the following steps:

i) measuring the expressions of NKp30a, NKp30b, NKp30c, NKp44b and NKp44c in a biological sample of said subject;
if the expressions of NKp30a, NKp30b, and NKp44b are the three highest among said group, then said subject has a responsive profile;
ii) mixing the biological sample of step i) with a composition comprising TGF-β, IL-15 and optionally IL-18; and
iii) reintroducing the mixture obtained at the end of step ii) into the subject.

### DETAILED DESCRIPTION OF THE INVENTION

The invention relates to a method for monitoring the immunological profile of a subject in need thereof (“monitoring method of the invention”), comprising measuring the expressions of each member of the group consisting of NKp30a, NKp30b, NKp30c, NKp44b and NKp44c, in a biological sample of said subject,

wherein:
if the expressions of NKp30a, NKp30b, and NKp44b are the three highest among said group, then said subject has a responsive profile; or
if the expressions of NKp30c and NKp44c are the two highest among said group, then said subject has an unresponsive profile.

The inventors have indeed identified two NK cell subsets (peripheral NK cells (pNK) and decidua basalis NK cells (dNK)) and their differential functioning. These two subsets express different NCR isoforms: whereas first trimester dNK cells predominantly express NKp30c, and NKp44c, pNK cells predominantly express NKp30a, NKp30b, and NKp44b, and optionally NKp44a.

This impacts cytokine microenvironment of said cells, and thus the consequent biological activity: dNK cells are unresponsive, thus are not activated, whereas pNK cells are responsive, thus induce an immune response.

NKp30a, NKp30b, and NKp30c are three among the 6 isoforms of the natural cytotoxicity triggering receptor 3 (NCR3 or NKp30). The gene NCR3 is transcribed in six different splice variants with NKp30a, NKp30b and NKp30c being the most abundant isoforms that have distinct function⁸. NKp30a and NKp30b convey stimulatory signals while NKp30c is rather immunosuppressive⁹. The protein sequence of said human receptor, and its isoforms, may be found in NCBI database with the following access numbers:

NKp30/NCR3 splice variant a: mRNA NM_147130.2, and protein_id: NP_667341.1, NKp30/NCR3 splice variant b: mRNA NM_001145466.1, and protein_id:NP_001138938.1, and
NKp30/NCR3 splice variant c: mRNA NM_001145467.1, and protein_id:NP_001138939.1.

NKp44a, NKp44b, and NKp44c are three among the 6 isoforms of the natural cytotoxicity triggering receptor 2 (NCR2 or NKp44). Said human receptor, and its isoforms, may be found in NCBI database with the following access numbers:

NKp44/NCR2 splice variant a: mRNA NM_001199510, and protein_id:NP_001186439.1,
NKp44/NCR2 splice variant b: mRNA NM_001199509.1, and protein_id:NP_001186438.1, and
NKp44/NCR2 splice variant c: mRNA NM_004828.3, and protein_id:NP_004819.2.

Finally, there is a natural cytotoxicity triggering receptor 1 (NCR1 or NKp46), corresponding to NM_004829.6 for splice variant a (protein id: NP_004820.2), NM_001145458.2 for splice variant b (protein id: NP_001138930.2) and NM_001242356.2 for splice variant c (protein id: NP_001229285.1), in NCBI database. NKp46/NCR1 and NKp30/NCR3 are expressed on resting and activated NK cells while NKp44/NCR2 is expressed only on activated NK cells⁴⁻⁷.

The method of the invention is for monitoring the immunological profile of a subject in need thereof.

The term “subject” as used herein refers to a mammalian, such as a rodent (e.g. a mouse or a rat), a feline, a canine or a primate. In a preferred embodiment, said subject is a human subject.

The subject according to the invention can be a healthy subject or a subject suffering from a given disease.

Preferably, said subject is grafted with cells or organ, or suffers from a viral infection or a cancer.

In a preferred embodiment, said subject is grafted with cells or organ. Thus, the subject may be grafted with stem cells, in particular allogeneic cardiac stem cells. The subject may also have undergone a bone marrow transplant.

In a preferred embodiment, said subject suffers from a viral infection, which is an infection caused by a virus selected from the group consisting of HIV, hepatitis E virus, hepatitis C virus, cytomegalovirus, Epstein-Barr virus and influenza viruses.

In another preferred embodiment of the invention, said subject suffers from a cancer, which is selected from the group comprising, but not limited to (preferably consisting of), melanoma, colon cancer, renal cancer and haematological malignancies such as leukemias, lymphomas and multiple myeloma.

The expression “monitoring the immunological profile” means evaluating changes in NKp30/NCR3 and NKp44/NCR2 splice variant expressions. The monitoring of the immunological profile of a given subject allows monitoring the NK cell activity of said subject.

According to the monitoring method of the invention, for a given subject, if the expressions of NKp30a, NKp30b, and NKp44b are the three highest expressions among the expressions of NKp30a, NKp30b, NKp30c, NKp44b and NKp44c, then one can conclude that said subject has a responsive profile. A “responsive profile” means that the NK cells of said subject have a pNK cell behavior: as the activating receptor signal is dominant, this will result in NK cell activation. In such a case, then one can conclude that said subject has activated NK cells which induce an immune response.

According to the monitoring method of the invention, for a given subject, if the expressions of NKp30c and NKp44c are the two highest expressions among the expressions of NKp30a, NKp30b, NKp30c, NKp44b and NKp44c, then one can conclude that said subject has an unresponsive profile. An “unresponsive profile” means that the NK cells of said subject have a dNK cell behavior: as the inhibitory signal is dominant, this will result in an inhibition of NK cell activity. In such a case, then one can conclude that said subject has non-activated NK cells.

Particularly, according to the monitoring method of the invention, a “responsive profile” (i.e. NK cells of a subject having a pNK cell behavior) expresses NKp30a, NKp30b, and NKp44b in respective amounts of at least 2 fold the respective amounts of NKp30a, NKp30b, and NKp44b expressed by an “unresponsive profile”.

Likewise, particularly, according to the monitoring method of the invention, an “unresponsive profile” (i.e. NK cells of a subject having a dNK cell behavior) expresses NKp30c and NKp44c in respective amounts of at least 2 fold the respective amounts of NKp30c and NKp44c expressed by a “responsive profile”.

Particularly, the invention relates to a method for monitoring the immunological profile of a subject in need thereof, comprising measuring the expressions of each member of the group consisting of NKp30a, NKp30b, NKp30c, NKp44b and NKp44c, in a biological sample of said subject,

wherein:
the expressions of NKp30a, NKp30b, and NKp44b being the three highest among said group are indicative that said subject has a responsive profile; or
the expressions of NKp30c and NKp44c being the two highest among said group are indicative that said subject has an unresponsive profile.

The group consisting of NKp30a, NKp30b, NKp30c, NKp44b and NKp44c is called “the group” in the present invention.

Preferably, the expression of NKp44a is further measured in the biological sample of the subject. In this case, if the expressions of NKp30a, NKp30b, NKp44b and NKp44a are the four highest among the expressions of NKp30a, NKp30b, NKp30c, NKp44a, NKp44b and NKp44c, then it is indicative that said subject has a responsive profile.

For example, when the subject has been grafted with cells or an organ, the monitoring method of the invention may be applied:
if the expressions of NKp30a, NKp30b, and NKp44b for said subject are the three highest among the group, then the subject has a responsive profile. In this case, immunosuppressive drugs may be administered to said subject in order to inhibit the activation of the NK cells, which may be responsible for a graft-vs-host disease (GvHD);
if the expressions of NKp30c and NKp44c for said subject are the two highest among the group, then the subject has an unresponsive profile. This means that the graft is well-tolerated by the subject.

According to a further example, when the subject suffers from a viral infection or cancer, the monitoring method of the invention may be applied:
if the expressions of NKp30a, NKp30b, and NKp44b for said subject are the three highest among the group, then the subject has a responsive profile. This means that the immune system of the subject is active against the viral infection or cancer;
if the expressions of NKp30c and NKp44c for said subject are the two highest among the group, then the subject has an unresponsive profile. In this case, immunostimulatory drugs may be administered to said subject in order to treat the viral infection or cancer.

The biological sample of the monitoring method of the invention is preferably a biopsy or a blood sample. The term “blood sample” as used herein preferably refers to a crude blood specimen which has been isolated from a subject and collected in tubes or other containers containing an appropriate anti-coagulant (e.g., lithium heparin or sodium citrate). The blood sample is preferably unfractionated whole blood and contains plasma and blood cells (red blood cells, white blood cells). It may be a freshly isolated blood sample (<48 h) or a blood sample which has been obtained previously and kept frozen until use.

The expressions of NKp30a, NKp30b, NKp30c, NKp44b and NKp44c, and optionally NKp44a, preferably correspond to the amounts of the corresponding mRNA or proteins.

The amount of corresponding mRNA may be measured starting from total RNAs. Total RNAs can be easily extracted from the biological sample. For instance, the biological sample may be treated prior to its use, e.g. in order to render nucleic acids available. Techniques of cell or protein lysis, concentration or dilution of nucleic acids, are known by the skilled person.

The extracted mRNA may then be subjected to coupled reverse transcription and amplification, such as reverse transcription and amplification by polymerase chain reaction (RT-PCR), using specific oligonucleotide primers that enable amplification of each of the NCR isoforms. Said primers are particularly listed in Table 1. Preferably quantitative or semi-quantitative RT-PCR is used. Real-time quantitative or semi-quantitative RT-PCR is particularly advantageous. Extracted mRNA may be reverse-transcribed and amplified, after which amplified sequences may be detected by hybridization with a suitable probe or by direct sequencing, or any other appropriate method known in the art. Other methods of amplification include ligase chain reaction (LCR), transcription-mediated amplification (TMA), strand displacement amplification (SDA) and nucleic acid sequence based amplification (NASBA).

Alternatively, the amount of corresponding proteins may be measured. The methods for measuring such an amount comprise contacting the biological sample with a binding partner capable of selectively interacting with one of the NCR isoforms present in the sample. The binding partner is generally an antibody that may be polyclonal or monoclonal, preferably monoclonal.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

The monitoring method of the invention may be performed on a given subject over a time period of from some days (i.e. from 1 to 15 days) to many months (i.e. from 1 to 24 months).

Particularly, the monitoring method of the invention may be performed before and after treatment of a given subject. The monitoring method of the invention indicates whether the subject presents a responsive or unresponsive profile. Thus, by comparing the obtained data before and after treatment, it may be concluded about the efficiency of the treatment.

The present invention also relates to a method for treating a subject in need thereof, preferably a subject grafted with cells or organ, comprising the following steps:

i) measuring the expressions of each member of the group consisting of NKp30a, NKp30b, NKp30c, NKp44b and NKp44c in a biological sample of said subject; wherein if the expressions of NKp30a, NKp30b, and NKp44b are the three highest among said group, then said subject has a responsive profile; and
ii) subsequently treating said patient with an immunosuppressive drug.

Said immunosuppressive drug may be chosen from folic acid analogues, antibodies and drugs acting on immunophilins. Preferably, the folic acid analogue is methotrexate. Preferably, the drug acting on immunophilins is chosen from ciclosporin, tacrolimus and sirolimus.

The present invention also relates to a method for converting a sample of NK cells having a responsive profile into NK cells having an unresponsive profile, comprising a step of mixing said sample with a composition comprising TGF-β and IL-15.

Indeed, as shown in the example, said composition is able to convert the phenotype pNK cells into the one of dNK cells. Thus, said composition is able to convert NK cells with a responsive profile into NK cells with an unresponsive profile.

Again, a “responsive profile” means that the NK cells are activated and induce an immune response. An “unresponsive profile” means an inhibition of NK cell activity. Preferably, the composition further comprises IL-18. Preferably, the composition comprises:

a) 1.5 to 4 ng/ml of TGF-β;
b) 5 to 20 ng/ml of IL-15; and
c) optionally 5 to 20 ng/ml of IL-18.

Thus, for example in case of a subject grafted with cells or organ, said composition may be used as a potent treatment for inactivating the immune system and avoiding a GvHD. The present invention relates to a method for treating a subject in need thereof, preferably a subject grafted with cells or organ, comprising the following steps:

i) measuring the expressions of each member of the group consisting of NKp30a, NKp30b, NKp30c, NKp44b and NKp44c in a biological sample of said subject;
ii) selecting the subject for which the expressions of NKp30a, NKp30b, and NKp44b are the three highest among the group;
iii) for the selected subject of step ii), mixing the biological sample of step i) with a composition comprising TGF-β, IL-15 and optionally IL-18; and
iv) reintroducing the mixture obtained at the end of step iii) into the subject.

Finally, the present invention relates to a method for treating a subject in need thereof, preferably a subject grafted with cells or organ, comprising the following steps:

i) measuring the expressions of each member of the group consisting of NKp30a, NKp30b, NKp30c, NKp44b and NKp44c in a biological sample of said subject;
ii) selecting the subject for which the expressions of NKp30a, NKp30b, and NKp44b are the three highest among the group; and
iii) administering a composition comprising TGF-β, IL-15 and optionally IL-18, to the selected subject of step ii).

The invention will be further described by the following examples, which are not intended to limit the scope of the protection defined by the claims.

### FIGURE LEGENDS

FIG. 1. dNK cells and pNK cells differentially express NCRs isoforms.

mRNA were extracted from freshly-isolated dNK and pNK cells that were purified from the same donor. Relative expression of the three NKp30 (a, b, c) and NKp44 (d, e, f) splice variants as determined by quantitative reverse transcription PCR (qRT-PCR) analysis. (a, d) Relative mRNA expression. (b, e) mRNA ratios calculated for each cell type. (c, f) mRNA relative expression ratios between dNK and pNK cells. Data are representative of ten independent donors. Bar graphs are mean±s.e.m. *P<0.05 and **P<0.01, ns: not significant.

FIG. 2. dNK and pNK cells are differentially activated after NCR cross-linking.

Freshly-isolated dNK and IL15-pNK cells were stimulated for four hours (a) with a single or a combination of two specific mAb, used as ligands. NK cell degranulation was assessed by quantification of CD107a cell surface expression using flow cytometry on CD3^{neg}CD56^{pos} cells. (b) Percentage of freshly-isolated dNK cells and (c) pNK cells showing CD107a surface expression. Results presented as mean values±s.e.m. from 4 independent experiments. *P<0.05, **P<0.01, ***P<0.001, ns: not significant.

FIG. 3. Decidual cytokine environment impacts pNK cell NCRs isoform expression.

Comparative expression of (a) NKp30a, (b) NKp30b, and (c) NKp30c mRNA isoforms in pNK cells cultured in media supplemented with IL15, IL18 and TGF-β, alone or in combination, relative to expression in pNK cells cultured in complete media. Bar graphs represented mean values±s.e.m. from four independent experiments. (d) Comparative expression of NKp44a, (e) NKp44b, and (f) NKp44c mRNA isoforms in pNK cells cultured in media supplemented with IL15, IL18 and TGF-β alone or in combination, relative to expression in pNK cells cultured in complete media. Bar graphs represented mean±s.e.m. from four independent experiments. (g) Fold induction of NKp30 and (h) NKp44 mRNA isoforms in six days cultured pNK cells relative to expression in pNK cells cultured in complete media. *P<0.05, **P<0.01, ***P<0.001, ns: not significant.

FIG. 4. NCR expression is modulated by pregnancy cytokines cocktail.

Mean Fluorescence Intensity (MFI) from four independent donors after six days culture with cytokine cocktail. Graphs represent freshly isolated dNK cells, pNK cells cultured in complete media or pNK cells in media supplemented with cytokine cocktail. Data on the graphs represent mean value±s.e.m. *P<0.05, **P<0.01, ***P<0.001, ns: not significant.

FIG. 5. Decidual cytokine environment impacts pNK cell function.

(a) CD107a cell surface expression on 6-days cultured pNK cells and freshly isolated dNK cells after 4 hours of NCR-ligation. NK cells were stimulated with anti-NKp30, -NKp44, -NKp46 antibodies or Isotype matched controls. Cell degranulation (CD107a expression) was assessed by flow cytometry on CD3^{neg}CD56^{pos} cells. Representative graphs of 6 independent experiments are presented. (b) Secreted TNF-α (c), IFN-γ (d), and VEGF-A (e) by 6-days cultured pNK cells and freshly isolated dNK cells was measured by multiplexed assay after 18 hours NKp30-, NKp44- or NKp46-ligation. Supernatants were collected from 3 independent experiments. Values represent mean±s.e.m. of triplicates within the same experiment. *P<0.05, **P<0.01, ***P<0.001.

### EXAMPLE 1: NKP30/NCR3 AND NKP44/NCR2 MICROENVIRONMENT-INURED ALTERNATIVE SPLICED VARIANTS DELINEATE DISTINCT NK CELL SUBSETS ORCHESTRATING THEIR FUNCTION

### Materials and Methods

### Ethics Statement

Informed consents were signed before samples were taken (Agence de la Biomédecine, PFS08-022, France).

Cell Purification

First-trimester decidua basalis (8-12 weeks of pregnancy) were obtained after elective termination of pregnancy as previously describes¹³. decidua basalis samples were minced and collagenase IV treated (Sigma-Aldrich, France). dNK cells were purified from non-adherent cell fraction using MACS negative selection kits (Miltenyi Biotec, France). pNK cells were isolated from healthy blood donors and stimulated or not with 10 ng/ml of IL15 overnight. More than 98% of purified cells are CD3^{neg}CD56^{pos}.

qRT-PCR

Total cellular RNA was isolated from dNK or pNK cells using RNeasy kit (Qiagen, France). First-strand cDNA was synthesized from 1 μg of total RNA using SuperScript III reverse transcriptase and random primers according to manufacturer's procedures (Life Technologies, France). PCR primers for NCR3⁹, NCR2 and the β-actin housekeeping transcripts were designed using NCBI primer blast (Table 1 below).

TABLE 1
Sequences of forward and reverse primers used for
qRT-PCR analyses. Primers were designed using
NCBI Blast and purchased from Sigma (Sigma,
France).

| Name | | Sequence |
|---|---|---|
| actin | Forward | 5′-CAAACATGATCTGGGTCATCTTCTC-3′ |
| | | (SEQ ID NO: 1) |
| actin | Reverse | 5′-GCTCGTCGTTCGACAACGGCT-3′ |
| | | (SEQ ID NO: 2) |
| NKp30/NCR3 | Forward | 5′-TTTCCTCCATGACCACCAGG-3′ |
| | | (SEQ ID NO: 3) |
| NKp30/NCR3 | Reverse | 5′-TTCTTGGACCTTTCCAGG-3′ |
| (a) | | (SEQ ID NO: 4) |
| NKp30/NCR3 | Reverse | 5′-CGGAGAGAGTAGATTTGGCATATT-3′ |
| (b) | | (SEQ ID NO: 5) |
| NKp30/NCR3 | Reverse | 5′-TTCCCATGTGACAGTGGCATT-3′ |
| (c) | | (SEQ ID NO: 6) |
| NKp44/NCR2 | Forward | 5′-AAGCCCCTGAGTCTCCATCT-3′ |
| (a) | | (SEQ ID NO: 7) |
| NKp44/NCR2 | Reverse | 5′-GTTTTCCACCATATGTCCCCC-3′ |
| (a) | | (SEQ ID NO: 8) |
| NKp44/NCR2 | Forward | 5′-TTCACAGACCCAGACCCAGAG-3′ |
| (b) | | (SEQ ID NO: 9) |
| NKp44/NCR2 | Reverse | 5′-AGGACGGGTGTGAAGGGACA-3′ |
| (b) | | (SEQ ID NO: 10) |
| NKp44/NCR2 | Forward | 5′-GTCCCTTCACAGCCACAGAA-3′ |
| (c) | | (SEQ ID NO: 11) |
| NKp44/NCR2 | Reverse | 5′-GAGACCTCCCTTGATGCTGC-3′ |
| (c) | | (SEQ ID NO: 12) |

The qRT-PCR data were determined and normalized to the actin housekeeping gene according to the standard 2^{-ΔCt} method.

### Immunoblotting

NK cells were stimulated for 20 min through receptor cross-linking on anti-NKp30-(clone-210847), anti-NKp44- (polyclonal goat IgG) or anti-NKp46-specific antibodies (clone-195314) coated plates. Cells were lysed in sample buffer (1% NP40, 20 mM HEPES (pH 7.9), 10 mM KCl, 1 mM EDTA, 1 mM PMSF, 1% glycerol and cocktail of proteases and phosphatases inhibitors). 8 μg of proteins separated on 12% SDS-PAGE were electrotransferred to Immobilon membranes, immunoblotted with anti-phosphotyrosine antibody (4G10R-Platinum, Millipore) and normalized to β-actin (MAB1501, clone-C4). Immunoreactivity was detected by chemiluminescence autoradiography (Sigma, Germany).

### Degranulation Assay

For degranulation assay, NK cells were stimulated through receptor ligation using antibody-coated tissue culture plates (10 μg/ml). Cells were stained with fluorochrome-conjugated anti-human CD107a (BD-Pharmingen) or isotype matched control then analyzed by Flow Cytometry. Histograms were obtained by applying a gate on CD3^{neg}CD56^{pos} cells and analyzed using FlowJo software 7.6.5.

NK cells phenotype pNK cells were cultured in the presence of indicated cytokines (2.5 ng/ml of TGF-β and 10 ng/ml of IL15 or IL18) for 6 days. Media were refreshed every 72 hours. Cells were immunostained with fluorochrome-conjugated antibodies: anti-CD56-APC, anti-CD3-PE-Cy7, anti-CD16-PE, anti-CD69-FITC, anti-NKG2D-PE, anti-NKG2A-PE, anti-NKp30-PE, anti-NKp44-PE, anti-NKp46-PE or anti-NKG2C-FITC (BD-Pharmingen). Histograms were obtained by applying a gate on CD3^{neg}CD56^{pos} cells and analyzed using FlowJo software 7.6.5.

### Confocal Microscopy

NK cells stimulated for 20 min on anti-NKp30, anti-NKp44, anti-NKp46 or anti-NKG2A coated glass-coverslips. Cells were paraformaldehyde fixed and stained with anti-perforin and anti-tubulin antibodies as previously described¹⁶. Filamentous actin cytoskeleton was visualized with AlexaFluor-conjugated phalloidin and nuclei stained with DAPI. Immune synapses (IS) were analyzed using Zeiss LSM710 confocal microscope (Carl Zeiss, Germany). Images were processed using ImageJ software.

### Mutiplex Cytokine and Chemokine Array

pNK cells were cultured in the presence of indicated cytokine (2.5 ng/ml of TGF-β and 10 ng/ml of IL15 or IL18) for 6 days. Cultured-pNK or freshly isolated dNK cells were stimulated through NCR-ligation for 18 hours. Cytokines, chemokines and growth factors levels were measured in culture supernatants by 7-multiplexed Affymetrix cytokine assay (TNF-α, IFN-γ, VEGF-A, CXCL8/IL-8, CCL3/MIP-1α, CCL4/MIP-1β and CXCL10/IP-10) according to the manufacturer's procedures (Procarta/eBioscience, France).

### Statistical Analysis

Comparisons among independent groups were made using a 2-tailed Student t test. Data are expressed as mean value±standard error of the mean (s.e.m.). P values less than 0.05 were considered significant. * P<0.05, ** P<0.01, *** P<0.001.

### Results

dNK and pNK Cells Display Differential Expression of NKp30 and NKp44 Splice Variants

NCR-engagement triggers different effector functions in dNK and pNK cells¹³. Therefore, the inventors investigated whether alternatively spliced variants of NCR might individualize these two NK cells subsets in a cohort of dNK and pNK cells from the same donors.

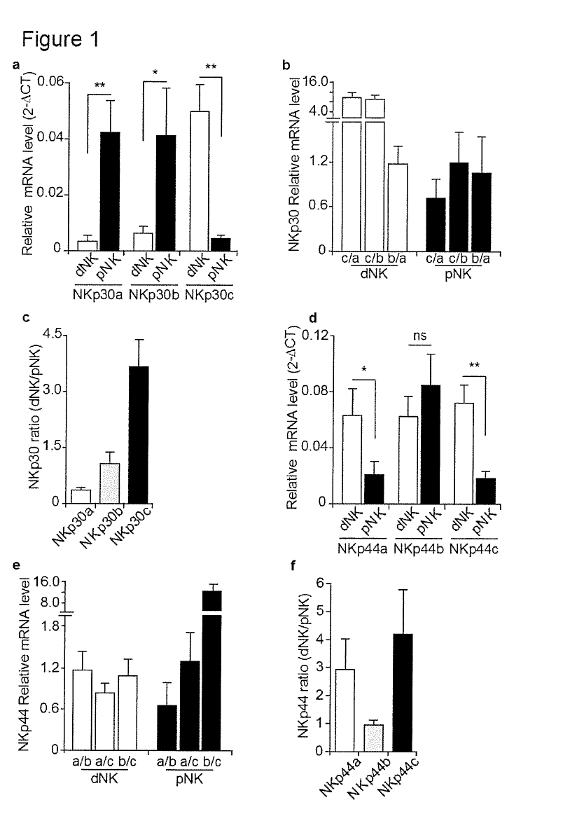

Consistent with previous report⁹, pNK cells displayed predominant expression of NKp30a and NKp30b mRNA but almost no NKp30c mRNA (FIG. 1a). In contrast, dNK cells significantly expressed lower amounts of NKp30a (p=0.0047) and NKp30b (p=0.05) mRNA but very high levels of NKp30c mRNA (p=0.0034) (FIG. 1a). Differences between dNK cells and pNK cells were further highlighted by the relative mRNA expression ratio (FIG. 1b). dNK cells express 8- to 10-fold higher amounts of NKp30c, while they express NKp30a and NKp30b at significantly lower levels. Compared to pNK cells, dNK cells express at least 3.5-fold more NKp30c (FIG. 1c). On the other hand, dNK cells displayed similar expression levels of all three NKp44 mRNAs while pNK cells exclusively expressed NKp44b mRNA (FIG. 1d). Relative ratios further demonstrated that freshly isolated pNK cells express 12-fold more NKp44b than NKp44c mRNAs (FIG. 1e). dNK cells express at least 3- and 4-time higher quantities of NKp44a and NKp44c mRNAs than pNK cells (FIG. 1f). Thus, dNK cells predominantly express NKp30c, NKp44a and NKp44c whereas pNK cells mainly express NKp30a, NKp30b and NKp44b. This differential expression of NKp30 and NKp44 isoforms could individualize these two NK cells subsets at the molecular level.

Differential Expression of NCRs Splice Variants Impacts NK Cell Cytotoxicity

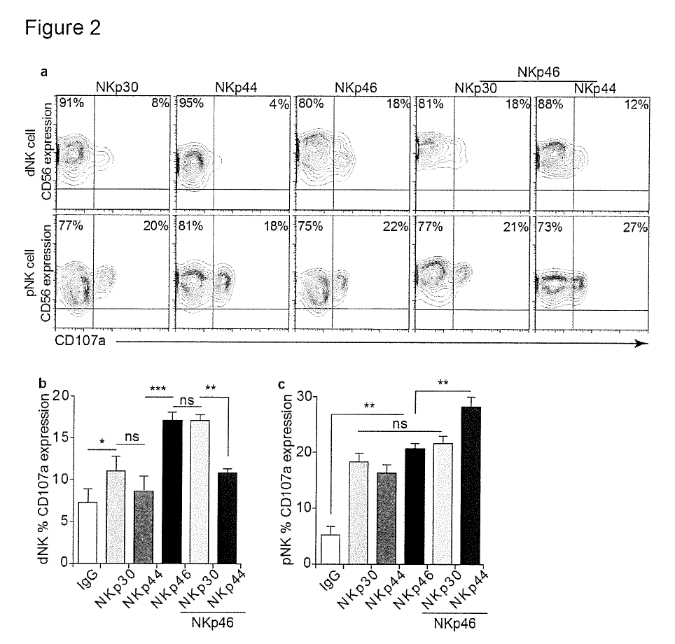

To examine the relevance of this dNK and pNK molecular individualization, the inventors investigated the impact of differential expression of NCR splice variants on cells effector functions. The inventors monitored cellular degranulation of dNK and pNK cells upon their NCR-ligation as readout for lytic activity. CD3^{neg}CD56^{pos} pNK and dNK cells were activated for 4 hours with anti-NCR antibodies and analyzed for cell surface expression of CD107a degranulation marker (FIG. 2). Ligation of NKp30 receptor but not NKp44, IgG isotype matched control (FIG. 2) or NKG2A inhibitory receptor (data not shown) on dNK cells resulted in a small increase of CD107a expression (P=0.05), while ligation of NKp46 induced dNK cell degranulation (FIG. 2a,b). Co-engagement of NKp44 and NKp46 receptors resulted in more than 30% decrease in NKp46-induced degranulation in dNK cells (P=0.0012) while co-ligation of NKp30 had no effect (FIG. 2a,b). In line with previous reports²³⁻²⁵, NKp30-, NKp44-, or NKp46-ligation resulted in significant increase of CD107a expression on pNK cells (FIG. 2a,c). Simultaneous NKp44- and NKp46-ligation showed an incremental effect on the ability of pNK cells to degranulate while co-engagement of NKp30 and NKp46 had no impact on NKp46-induced degranulation (FIG. 2a,c).

Together, these data demonstrate that only NKp46-ligation triggers robust lytic granule exocytosis in dNK cells. While NKp30 prompted mainly by isoform c induces modest degranulation in these cells, NKp44 prompted mainly by isoforms a and c acts rather as an inhibitory receptor. In contrast, all three NCRs similarly favor pNK cell degranulation. Thus, the molecular individualization of dNK and pNK cells is biologically or functionally relevant.

NCRs Splice Variants Impact NK Cell Immune Synapse (IS) Formation

Cytotoxic activity of NK cells is a dynamic process orchestrated in different steps. Receptor ligation leads to recruitment and activation of signaling pathways. To validate the relevance of observed molecular individualization of dNK and pNK subsets, the inventors sought to provide mechanistic insights into the differential functions of NCR isoforms. First the inventors analyzed tyrosine phosphorylation after NCR-ligation. Upon NKp30- or NKp44-ligation tyrosine phosphorylation patterns were quite different between the two NK cell populations. However, phosphorylation patterns were similar after NKp46-ligation on dNK and pNK cells (data not shown). These differences suggest that the engagement of the same NCR would trigger the recruitment of different signaling pathways in dNK or pNK cells.

Immune synapse formation is associated with cortical actin remodeling. The organization of actin-containing micro-domains at the vicinity of the MTOC serves as a docking site for lytic granules²⁶. To provide further mechanistic insights, the inventors then examined the impact of differential tyrosine phosphorylation patterns on the molecular organization of IS after single NCR-ligation on pNK and dNK cells (data not shown). NKp30-ligation resulted in a rapid reorganization of F-actin enriched cytoskeleton and MTOC and polarization of lytic granules in more than 45% of pNK cells (data not shown). By contrast, less than 20% of dNK cells showed polarized lytic granules after NKp30-ligation. Similar to NKp30, NKp44-ligation induced organized IS in more than 50% of pNK cells while only minor effects were seen in dNK cells (data not shown). Finally, activation through NKp46-ligation had similar effects on pNK or dNK cells with almost 50% of cells sharing features of cytolytic IS (data not shown). Similar to IgG control (data not shown), less than 20% of cells showed organized IS after ligation of control NKG2A inhibitory receptor (data not shown).

Overall, these data demonstrate that recruitment of different signaling pathways by NKp30 and NKp44 in dNK and pNK cells significantly impacts IS formation and cytotoxic activity of these two NK cells subsets. In line with the cytotoxic activity, all three NCRs favor pNK cell lytic IS formation while only NKp46-ligation triggers lytic IS in dNK cells. Thus, differential NCR splice variants expression by dNK and pNK is biologically and functionally relevant, which could probably contribute to promote their distinct functioning.

Decidual Microenvironment Modulates the mRNA Expression of pNK Cell NCR Isoforms

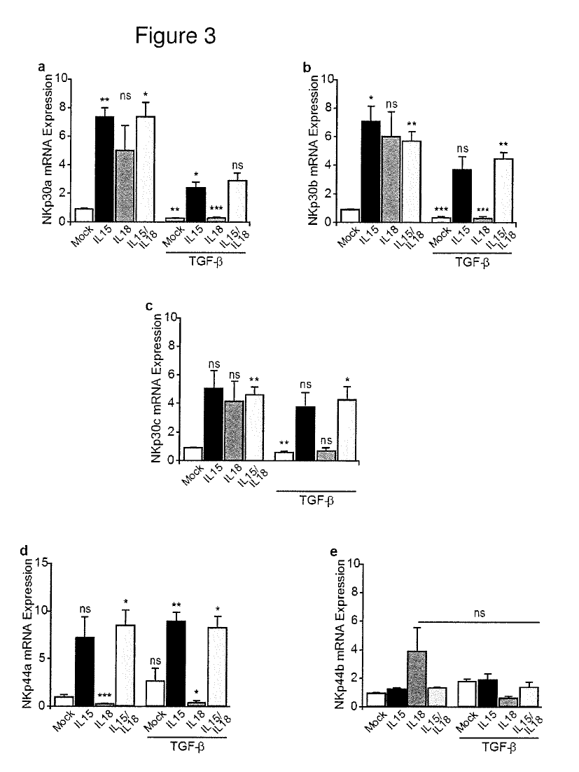

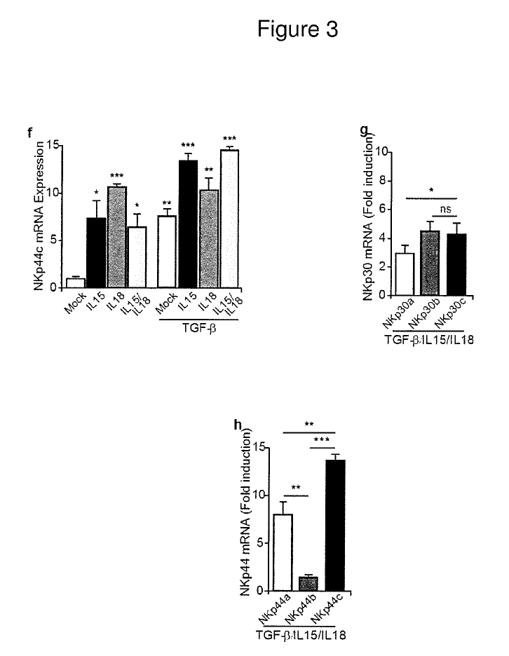

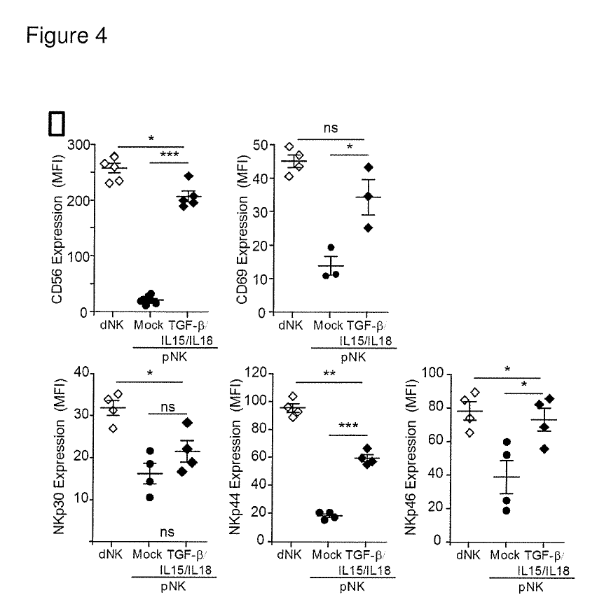

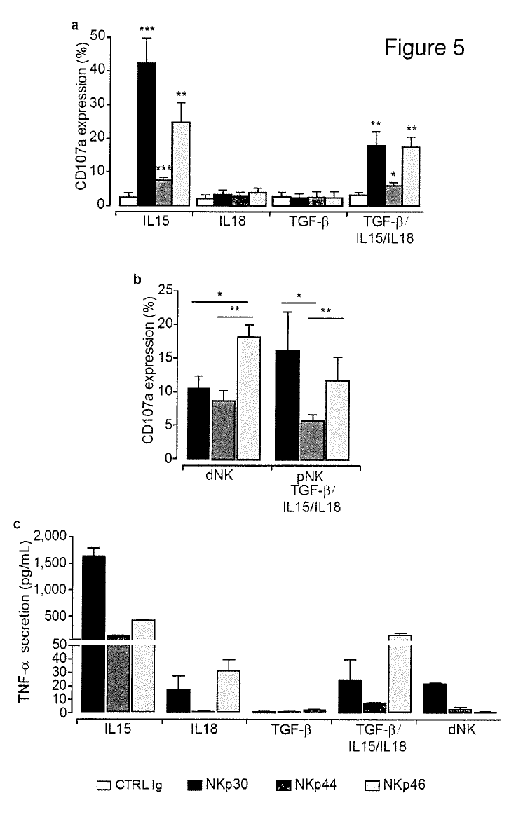

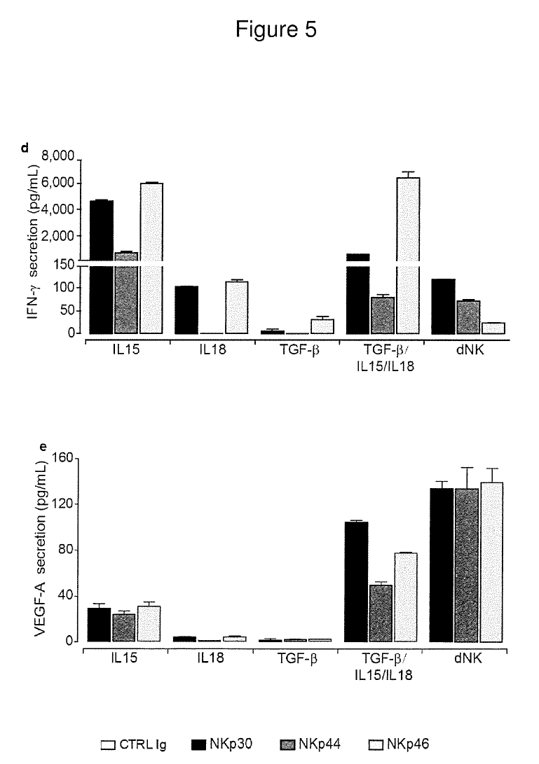

The above findings promoted us to investigate the elements that might contribute to sculpturing the expression of NCRs splice variants in NK cells subsets. dNK and pNK cells operate within distinct microenvironment. dNK operate within maternal endometrium enriched with immunomodulatory proteins (TGF-β) and pro-inflammatory cytokines such as IL15 and IL18 that are produced by the decidual stroma hosting fetal trophoblast²⁷⁻²⁹. Therefore, the inventors analyzed the potential role of these cytokines in leading the expression of NCR splice variants in pNK toward the expression profile of dNK cells. pNK cells were cultured in the presence of TGF-β/IL15/IL18 cocktail as well as other cytokine combinations as indicated (FIG. 3) and their mRNAs levels of NKp30 and NKp44 transcripts were evaluated. Compared to untreated-pNK cells, IL15 induced significant increase of NKp30a and NKp30b mRNA (P=0.0035 and 0.0142 respectively). Increases were also observed for NKp30c mRNA but did not reach significant levels due to variations amongst individuals. Treatment with TGF-β induced significant decrease in the relative mRNA level of all three transcripts (FIG. 3a,b,c). The presence of IL15 tempers the TGF-β effect although this does not reach significance for NKp30b and NKp30c splice variants. IL18 alone significantly increases the basic level of NKp30a splice variant mRNA (P=0.029) but its presence does not override TGF-β effect (FIG. 3a,b,c). Finally, the TGF-β/IL15/IL18 cocktail induces significant increases of the basic level of all three NKp30 transcripts (P=0.0254, 0.009 and 0.0325 respectively) (FIG. 3a,b,c). Moreover, when compared to each other, increases of NKp30b (4.5-fold) and NKp30c (4.3-fold) induced by the TGF-β/IL15/IL18 cocktail dominate that of NKp30a (2.9-fold, P=0.014) (FIG. 3g).

Next, the inventors examined the expression of NKp44 splice variants. The presence of IL15, IL18 or TGF-β alone or in combination induces significant changes in the expression level of NKp44a and NKp44c without affecting the expression of NKp44b mRNA (FIG. 3d,e,f). IL15 increases the basal level of NKp44a, whereas IL18 rather down regulates NKp44a. IL15/IL18 in the presence or absence of TGF-β induces the highest up-regulation of this mRNA (P=0.015) (FIG. 3d,e,f). IL18 is more effective in increasing the basic level of NKp44c isoform (P=0.0006) and again IL15/IL18 in the presence (P=0.0018) or absence (P=0.0346) of TGF-β significantly up-regulates its expression (FIG. 3f). Compared to NKp44b, TGF-β/IL15/IL18 induces seven-fold increase of NKp44a (14-fold, P=0.0195) and significantly higher increase of NKp44c (P=0.0005) (FIG. 3h).

Thus, a microenvironment rich in TGF-β/IL15/IL18 combination shifts the expression of NKp30 and NKp44 splice variants in pNK cells toward that of dNK cells. The data attribute an important role for cytokinic microenvironment in sculpturing and maintaining molecular individualization of NK cells subsets at least in terms of NKp30 and NKp44 splice variants mRNA transcription in pNK and dNK cells.

Decidual Microenvironment Impacts pNK Cell Phenotype

To support that microenvironment-inured NKp30/NCR3 and NKp44/NCR2 alternative spliced variants polarize NK cells subsets, the inventors analyzed the potential role of TGF-β/IL15/IL18 cocktail and other cytokine combinations in modulating NK cell phenotype.

In the presence of TGF-β/IL15/IL18 and consistent with previous reports³⁰, pNK cells up-regulated their CD56 expression and more than 98% of the cells become CD56^{bright}. Cells showed significant increases of Mean Fluorescence Intensity (MFI) 200.6±9.6 instead of 20.7±2.6 for cells cultured in medium (P<0.0001), reaching levels observed for dNK cells (259±8.4) (FIG. 4). Lower CD56 MFI increases were also observed when pNK cells were cultured with IL15 (117±12, P=0.0129) or TGF-β/IL15 (164±4, P=0.001). The presence of IL15 also significantly increased CD69 expression, which is barely expressed on freshly isolated pNK cells (FIG. 4) (P=0.0002 for IL15 alone, P=0.006 for IL15/IL18, P=0.005 for TGF-β/IL15 or P=0.001 for TGF-β/IL15/IL18). More than 40% of cytokine-cultured pNK cells expressed CD69 with MFI of 38.4±5 (FIG. 4) reaching the level of dNK cells (45±5). Furthermore, the presence of IL15 allowed the maintenance of CD16 expression in more than 70% of cells whereas only minor changes were observed for other receptors including NKG2D, NKG2C and NKG2A (data not shown) controlling the specificity of observed results.

The inventors next analyzed NCRs expression on pNK cells. More than 60% of pNK cells express NKp30. Treatment with TGF-β/IL15/IL18 does not affect the expression level of NKp30, which is decreased under other culture conditions (FIG. 4). Treatment of pNK cells with IL15 alone or in combination with the other two cytokines induced NKp44 expression in 60% of cells (data not shown). MFI comparison showed very low variability amongst different donors, with TGF-13/IL15/IL18 treatment shifting pNK cells towards dNK cell phenotype (FIG. 4). Finally, the presence of IL15 was able to up-regulate NKp46 MFI but addition of TGF-β had no effect. These changes were not due to cell proliferation or cell death. Only slight increases of proliferation were observed after six days of culture with IL15 and cell death was negligible.

Thus, similar to their effect on expression of NKp30 and NKp44 splice variants, TGF-β/IL15/IL18 cytokines sculpt the phenotype of pNK cells shifting it towards dNK cells phenotype. This finding supports that the phenotype and probably the effector functions of various NK cell subsets, namely dNK and pNK cells in this report, are commanded at least in part by their cytokine microenvironment.

Decidual Microenvironment Impacts pNK Cell Function

The inventors then tested whether microenvironment-inured NKp30/NCR3 and NKp44/NCR2 alternative changes in pNK cell phenotype would impact their effector functions. Cells were cultured in different cytokine combinations and their lytic function was assessed through analyses of CD107a expression after NCR-ligation by flow cytometry (FIG. 5a). While pNK cells cultured in medium show low capacity to degranulate, IL15-maintained pNK cells significantly degranulate upon NKp30-(42±7.6%), NKp46- (24.7±5.9%) and, to a much lesser extent, NKp44-ligation (8.1±0.5%). IL15/IL18 combination showed similar results to IL15 alone. Treatment with TGF-β did not affect basal level of CD107a expression in pNK cells. However, TGF-β/IL15 or TGF-β/IL15/IL18 significantly decreased the capacity of NKp30- and to lesser extent NKp44-stimulation to induce CD107a expression (17.8% and 5.7% respectively) but it did not blunt the response through NKp46 (FIG. 5a). These data indicate that pNK cells have normal capacities to degranulate upon NCR engagement and TGF-β does not affect the NCRs response in the same manner.

The inventors next compared cytokine secretion by pNK cells, maintained under different conditions, to that of dNK cells after NCR-ligation (FIG. 5). Cells were stimulated through NCR-ligation and supernatants were analyzed by multiplexed assay (TNF-α, IFN-γ, VEGF-A, CCL3, CCL4, CXCL8 and CXCL10). Medium-maintained pNK cells showed very low capacity to produce TNF-α, IFN-γ or VEGF-A but they produced substantial amounts CCL4 in response to NKp30- or NKp46-ligation. Only minor changes were observed for CCL3, CXCL8 and CXCL10. The presence of IL15 induced strong increases of TNF-α, IFN-γ, VEGF-A, CCL3, CCL4 and CXCL8 after NCR-ligation but did not affect the basal level of CXCL10 secretion (FIG. 5). IL18 alone had only minor effect on cytokine production and IL15/IL18 showed capacity similar to IL15 alone. TGF-β alone showed minor effects. Compared to IL15, TGF-β/IL15 or TGF-β/IL15/IL18 treatment strongly decreased the capacity of NKp30-ligation to induce high amount of TNF-α, IFN-γ and CCL3 (FIG. 5b,c) but increased VEGF-A production (FIG. 5d). Similar to NKp30, NKp44-ligation showed decrease in TNF-α, IFN-γ, CCL3 and CCL4 but did not affect the secretion of VEGF-A (FIG. 5). The addition of TGF-β did not impair NKp46-induced secretion of TNF-α, IFN-γ or CCL3 of IL15-cultured pNK cells (FIG. 5). Compared to IL15-treatment NKp46-ligation resulted in increased VEGF-A production in TGF-β/IL15- or TGF-β/IL15/IL18-treated cells (FIG. 5d).

Taken together, these data suggest that the combination of TGF-β, IL15 and IL18, results in pNK cells behaving similar to dNK cells since they secrete large amounts of VEGF-A and very low amounts of IFN-γ, TNF-α in response to NKp30- or NKp44-ligation. However, only minor changes are observed upon NKp46-ligation in TGF-β/IL15/IL18-treated cells.

### REFERENCES

1 Hudspeth, K., Silva-Santos, B. & Mavilio, D. Natural cytotoxicity receptors: broader expression patterns and functions in innate and adaptive immune cells. Frontiers in immunology 4, 69, doi:10.3389/fimmu.2013.00069 (2013).
2 Lanier, L. L. Up on the tightrope: natural killer cell activation and inhibition. Nature immunology 9, 495-502 (2008).
3 Moretta, A., Locatelli, F. & Moretta, L. Human NK cells: from HLA class I-specific killer Ig-like receptors to the therapy of acute leukemias. Immunological reviews 224, 58-69, doi:10.1111/j.1600-065X.2008.00651.x (2008).
4 Cantoni, C. et al. NKp44, a triggering receptor involved in tumor cell lysis by activated human natural killer cells, is a novel member of the immunoglobulin superfamily. The Journal of experimental medicine 189, 787-796 (1999).
5 Sivori, S. et al. p46, a novel natural killer cell-specific surface molecule that mediates cell activation. The Journal of experimental medicine 186, 1129-1136 (1997).
6 Vitale, M. et al. NKp44, a novel triggering surface molecule specifically expressed by activated natural killer cells, is involved in non-major histocompatibility complex-restricted tumor cell lysis. The Journal of experimental medicine 187, 2065-2072 (1998).
7 Pende, D. et al. Identification and molecular characterization of NKp30, a novel triggering receptor involved in natural cytotoxicity mediated by human natural killer cells. The Journal of experimental medicine 190, 1505-1516 (1999).
8 Hollyoake, M., Campbell, R. D. & Aguado, B. NKp30 (NCR3) is a pseudogene in 12 inbred and wild mouse strains, but an expressed gene in Mus caroli. Molecular biology and evolution 22, 1661-1672, doi:10.1093/molbev/msi162 (2005).
9 Delahaye, N. F. et al. Alternatively spliced NKp30 isoforms affect the prognosis of gastrointestinal stromal tumors. Nature medicine 17, 700-707 (2011).
10 Pessino, A. et al. Molecular cloning of NKp46: a novel member of the immunoglobulin superfamily involved in triggering of natural cytotoxicity. The Journal of experimental medicine 188, 953-960 (1998).
11 Fuchs, A., Cella, M., Kondo, T. & Colonna, M. Paradoxic inhibition of human natural interferon-producing cells by the activating receptor NKp44. Blood 106, 2076-2082 (2005).
12 Apps, R., Gardner, L., Traherne, J., Male, V. & Moffett, A. Natural-killer cell ligands at the maternal-fetal interface: UL-16 binding proteins, MHC class-I chain related molecules, HLA-F and CD48. Human reproduction (Oxford, England) 23, 2535-2548, doi:10.1093/humrep/den223 (2008).
13 El Costa, H. et al. Critical and differential roles of NKp46- and NKp30-activating receptors expressed by uterine NK cells in early pregnancy. J Immunol 181, 3009-3017 (2008).
14 Hanna, J. et al. Decidual NK cells regulate key developmental processes at the human fetal-maternal interface. Nature medicine 12, 1065-1074 (2006).
15 Jabrane-Ferrat, N. & Siewiera, J. The up side of decidual natural killer cells: new developments in immunology of pregnancy. Immunology 141, 490-497, doi:10.1111/imm.12218 (2014).
16 Siewiera, J. et al. Human cytomegalovirus infection elicits new decidual natural killer cell effector functions. PLoS pathogens 9, e1003257, doi:10.1371/journal.ppat.1003257 (2013).
17 Vacca, P. et al. Regulatory role of NKp44, NKp46, DNAM-1 and NKG2D receptors in the interaction between NK cells and trophoblast cells. Evidence for divergent functional profiles of decidual versus peripheral NK cells. International immunology 20, 1395-1405, doi:10.1093/intimm/dxn105 (2008).
18 Hanna, J. et al. CXCL12 expression by invasive trophoblasts induces the specific migration of CD16-human natural killer cells. Blood 102, 1569-1577 (2003).
19 Manaster, I. et al. Endometrial NK cells are special immature cells that await pregnancy. J Immunol 181, 1869-1876 (2008).
20 Vacca, P. et al. CD34+ hematopoietic precursors are present in human decidua and differentiate into natural killer cells upon interaction with stromal cells. Proceedings of the National Academy of Sciences of the United States of America 108, 2402-2407 (2011).
21 Moffett, A. & Loke, C. Immunology of placentation in eutherian mammals. Nature reviews 6, 584-594, doi:10.1038/nri1897 (2006).
22 Koopman, L. A. et al. Human decidual natural killer cells are a unique NK cell subset with immunomodulatory potential. The Journal of experimental medicine 198, 1201-1212 (2003).
23 Das, R. & Tripathy, A. Increased expressions of NKp44, NKp46 on NK/NKT-like cells are associated with impaired cytolytic function in self-limiting hepatitis E infection. Medical microbiology and immunology 203, 303-314, doi:10.1007/s00430-014-0338-1 (2014).
24 Sivori, S. et al. Involvement of natural cytotoxicity receptors in human natural killer cell-mediated lysis of neuroblastoma and glioblastoma cell lines. Journal of neuroimmunology 107, 220-225 (2000).
25 Wang, H., Zheng, X., Wei, H., Tian, Z. & Sun, R. Important role for NKp30 in synapse formation and activation of NK cells. Immunological investigations 41, 367-381, doi:10.3109/08820139.2011.632799 (2012).
26 Brown, A. C. et al. Remodelling of cortical actin where lytic granules dock at natural killer cell immune synapses revealed by super-resolution microscopy. PLoS biology 9, e1001152, doi:10.1371/journal.pbio.1001152 (2011).
27 Graham, C. H., Lysiak, J. J., McCrae, K. R. & Lala, P. K. Localization of transforming growth factor-beta at the human fetal-maternal interface: role in trophoblast growth and differentiation. Biology of reproduction 46, 561-572 (1992).
28 Kitaya, K. et al. IL-15 expression at human endometrium and decidua. Biology of reproduction 63, 683-687 (2000).
29 Tokmadzic, V. S. et al. IL-18 is present at the maternal-fetal interface and enhances cytotoxic activity of decidual lymphocytes. Am J Reprod Immunol 48, 191-200 (2002).
30 Keskin, D. B. et al. TGFbeta promotes conversion of CD16+ peripheral blood NK cells into CD16- NK cells with similarities to decidual NK cells. Proceedings of the National Academy of Sciences of the United States of America 104, 3378-3383 (2007).
31 Campbell, K. S., Yusa, S., Kikuchi-Maki, A. & Catina, T. L. NKp44 triggers NK cell activation through DAP12 association that is not influenced by a putative cytoplasmic inhibitory sequence. J Immunol 172, 899-906 (2004).
32 Kopcow, H. D. et al. Human decidual NK cells form immature activating synapses and are not cytotoxic. Proceedings of the National Academy of Sciences of the United States of America 102, 15563-15568 (2005).
33 Perez-Quintero, L. A. et al. EAT-2, a SAP-like adaptor, controls NK cell activation through phospholipase Cgamma, Ca++, and Erk, leading to granule polarization. The Journal of experimental medicine 211, 727-742, doi:10.1084/jem.20132038 (2014).
34 Cerdeira, A. S. et al. Conversion of peripheral blood NK cells to a decidual NK-like phenotype by a cocktail of defined factors. J Immunol 190, 3939-3948, doi:10.4049/jimmunol.1202582 (2013).
35 Dun, S. & Kindler, V. Implication of indolamine 2,3 dioxygenase in the tolerance toward fetuses, tumors, and allografts. Journal of leukocyte biology 93, 681-687, doi:10.1189/jlb.0.0712347 (2013).
36 Renaud, S. J., Macdonald-Goodfellow, S. K. & Graham, C. H. Coordinated regulation of human trophoblast invasiveness by macrophages and interleukin 10. Biology of reproduction 76, 448-454, doi:10.1095/biolreprod.106.055376 (2007).
37 Mandelboim, O. et al. Recognition of haemagglutinins on virus-infected cells by NKp46 activates lysis by human NK cells. Nature 409, 1055-1060 (2001).
38 Mavoungou, E., Held, J., Mewono, L. & Kremsner, P. G. A Duffy binding-like domain is involved in the NKp30-mediated recognition of Plasmodium falciparum-parasitized erythrocytes by natural killer cells. The Journal of infectious diseases 195, 1521-1531 (2007).
39 Vieillard, V., Strominger, J. L. & Debre, P. NK cytotoxicity against CD4+ T cells during HIV-1 infection: a gp41 peptide induces the expression of an NKp44 ligand. Proceedings of the National Academy of Sciences of the United States of America 102, 10981-10986 (2005).
40 Holder, K. A., Stapleton, S. N., Gallant, M. E., Russell, R. S. & Grant, M. D. Hepatitis C virus-infected cells downregulate NKp30 and inhibit ex vivo NK cell functions. J Immunol 191, 3308-3318, doi:10.4049/jimmunol.1300164 (2013).
41 Donatelli, S. S. et al. TGF-beta-inducible microRNA-183 silences tumor-associated natural killer cells. Proceedings of the National Academy of Sciences of the United States of America 111, 4203-4208, doi:10.1073/pnas.1319269111 (2014).
42 Arnon, T. I. et al. Inhibition of the NKp30 activating receptor by pp65 of human cytomegalovirus. Nature immunology 6, 515-523 (2005).
43 Furman, M. H., Dey, N., Tortorella, D. & Ploegh, H. L. The human cytomegalovirus US10 gene product delays trafficking of major histocompatibility complex class I molecules. Journal of virology 76, 11753-11756 (2002).
44 Laprevotte, E. et al. Recombinant human IL-15 trans-presentation by B leukemic cells from chronic lymphocytic leukemia induces autologous NK cell proliferation leading to improved anti-CD20 immunotherapy. J Immunol 191, 3634-3640, doi:10.4049/jimmunol.1300187 (2013).
45 Sargent, I. L., Borzychowski, A. M. & Redman, C. W. NK cells and human pregnancy—an inflammatory view. Trends in immunology 27, 399-404 (2006).
46 Hu, W., Wang, H., Wang, Z., Huang, H. & Dong, M. Elevated serum levels of interleukin-15 and interleukin-16 in preeclampsia. Journal of reproductive immunology 73, 166-171, doi:10.1016/j.jri.2006.06.005 (2007).
47 Peracoli, M. T. et al. Platelet aggregation and TGF-beta(1) plasma levels in pregnant women with preeclampsia. Journal of reproductive immunology 79, 79-84, doi:10.1016/j.jri.2008.08.001 (2008).
48 Pugliese, A., Beltramo, T., Todros, T., Cardaropoli, S. & Ponzetto, A. Interleukin-18 and gestosis: correlation with Helicobacter pylori seropositivity. Cell biochemistry and function 26, 817-819, doi:10.1002/cbf.1503 (2008).
49 Cooper, M. A. et al. Cytokine-induced memory-like natural killer cells. Proceedings of the National Academy of Sciences of the United States of America 106, 1915-1919, doi:10.1073/pnas.0813192106 (2009).
50 Lopez-Verges, S. et al. Expansion of a unique CD57(+)NKG2Chi natural killer cell subset during acute human cytomegalovirus infection. Proceedings of the National Academy of Sciences of the United States of America 108, 14725-14732 (2011).

1-4. (canceled)

5. Method according to claim 15, wherein said biological sample is a biopsy or a blood sample.

6. Method according to claim 15, wherein the subject is grafted with stem cells; or has undergone a bone marrow transplant.

7. Method according to claim 17, wherein said cancer is selected from the group consisting of melanoma, colon cancer, renal cancer and haematological malignancies.

8. Method according to claim 17, wherein said viral infection is an infection caused by a virus selected from the group consisting of HIV, hepatitis E virus, hepatitis C virus, cytomegalovirus, Epstein-Barr virus and influenza viruses.

9-11. (canceled)

12. Method for converting a sample of NK cells having a responsive profile into NK cells having an unresponsive profile, comprising a step of mixing said sample with a composition comprising TGF-β and IL-15.

13. Method according to claim 12, wherein the composition further comprises IL-18.

14. Method according to claim 12, wherein the composition comprises:
a) 1.5 to 4 ng/ml of TGF-β;
b) 5 to 20 ng/ml of IL-15; and
c) optionally 5 to 20 ng/ml of IL-18.

15. A method for treating a subject grafted with cells or organ, comprising the following steps:
i) measuring the expressions of the group of NKp30a, NKp30b, NKp30c, NKp44b and NKp44c in a biological sample of said subject;
ii) treating said subject with an immunosuppressive drug when the expressions of NKp30a, NKp30b, and NKp44b are the three highest among said group.

16. A method for treating a subject grafted with cells or organ, comprising the following steps:
i) measuring the expressions of the group of NKp30a, NKp30b, NKp30c, NKp44b and NKp44c in a biological sample of said subject;
ii) mixing the biological sample of step i) with a composition comprising TGF-β, IL-15 and optionally IL-18 when the expressions of NKp30a, NKp30b, and NKp44b are the three highest among said group; and
iii) introducing the mixture obtained at the end of step ii) into the subject.

17. A method for treating a subject suffering from a viral infection or cancer, comprising the following steps:
i) measuring the expressions of the group of NKp30a, NKp30b, NKp30c, NKp44b and NKp44c in a biological sample of said subject;
ii) treating said subject with an immunostimulatory drug when the expressions of NKp30c and NKp44c for said subject are the two highest among said group.

18. The method of claim 6, wherein the stem cells are allogeneic cardiac stem cells.

19. The method of claim 7, wherein the haematological malignancy is leukemia, lymphoma or multiple myeloma.


SEQUENCE LISTING (12 sequences)

SEQ ID NO: 1 - DNA, 25 bp, Artificial Sequence
  primer forward beta actin
caaacatgatctgggtcatcttctc

SEQ ID NO: 2 - DNA, 21 bp, Artificial Sequence
  primer reverse beta actin
gctcgtcgttcgacaacggct

SEQ ID NO: 3 - DNA, 20 bp, Artificial Sequence
  primer forward NKp30
tttcctccatgaccaccagg

SEQ ID NO: 4 - DNA, 18 bp, Artificial Sequence
  primer reverse NKp30a
ttcttggacctttccagg

SEQ ID NO: 5 - DNA, 24 bp, Artificial Sequence
  primer reverse NKp30b
cggagagagtagatttggcatatt

SEQ ID NO: 6 - DNA, 21 bp, Artificial Sequence
  primer reverse NKp30c
ttcccatgtgacagtggcatt

SEQ ID NO: 7 - DNA, 20 bp, Artificial Sequence
  primer forward NKp44a
aagcccctgagtctccatct

SEQ ID NO: 8 - DNA, 21 bp, Artificial Sequence
  primer reverse NKp44a
gttttccaccatatgtccccc

SEQ ID NO: 9 - DNA, 21 bp, Artificial Sequence
  primer forward NKp44b
ttcacagacccagacccagag

SEQ ID NO: 10 - DNA, 20 bp, Artificial Sequence
  primer reverse NKp44b
aggacgggtgtgaagggaca

SEQ ID NO: 11 - DNA, 20 bp, Artificial Sequence
  primer forward NKp44c
gtcccttcacagccacagaa

SEQ ID NO: 12 - DNA, 20 bp, Artificial Sequence
  primer reverse NKp44c
gagacctcccttgatgctgc